(19) United States
(12) Patent Application Publication
(10) Pub. No.: US 2025/0363656 A1
(43) Pub. Date: Nov. 27, 2025

(54) ENDOSCOPIC IMAGE PROCESSING DEVICE AND METHOD FOR OPERATING ENDOSCOPIC IMAGE PROCESSING DEVICE

(71) Applicant: OLYMPUS MEDICAL SYSTEMS CORP., Tokyo (JP)

(72) Inventors: Kumiko TAMURA, Tokyo (JP); Makoto KITAMURA, Tokyo (JP); Hiromu SUGITA, Tokyo (JP)

(73) Assignee: OLYMPUS MEDICAL SYSTEMS CORP., Tokyo (JP)

(21) Appl. No.: 19/297,269

(22) Filed: Aug. 12, 2025

Related U.S. Application Data
(63) Continuation of application No. PCT/JP2023/005323, filed on Feb. 15, 2023.

(51) Int. Cl. G06T 7/70; G06T 7/90; G08B 21/18

(52) U.S. Cl. CPC G06T 7/70; G06T 7/90; G08B 21/182; G06T 2207/10068

(57) ABSTRACT

One or more processors create a first image from a first signal, create a second image from a second signal, determine whether visibility of at least a part of the second image is equal to or higher than a second threshold, or lower than the second threshold, and create different notification information depending on a result of the determination of the visibility. The one or more processors create at least one kind of: the notification information for guiding to the second image when the visibility is equal to or higher than the second threshold; or the notification information for notifying that the second image is not suitable for viewing when the visibility is lower than the second threshold.

### CROSS-REFERENCE TO RELATED APPLICATION

This application is a continuation application of PCT/JP2023/005323 filed on Feb. 15, 2023, the entire contents of which are incorporated herein by this reference.

### FIELD

The present disclosure relates to an endoscopic image processing device for performing processing depending on visibility of at least a part of an image and a method for operating the endoscopic image processing device.

### BACKGROUND

Conventionally, endoscopes have acquired a normal light image by irradiating a subject with normal light such as white light. Furthermore, in some cases, endoscopes acquire a special light image by irradiating a subject with special light whose spectral distribution is different from that of white light. Note that acquiring the special light image is not limited to irradiating the subject with the special light. The special light image may be acquired by performing image processing, which is different from image processing for the normal light image, on a signal acquired by irradiating the subject with the normal light.

For example, Japanese Patent Application Laid-Open Publication No. 2022-63129 discloses a technique for displaying a normal light image and a special light image on one display included in an endoscope system.

In some cases, a special light image is effective for detecting a lesion candidate region (at least a part of an image) which is a candidate of a lesion region. Furthermore, for example, an artificial intelligence (AI) is sometimes used for detection of a lesion candidate region. A normal light image is suitable for thoroughly observing an entire screen. In view of this, during an operation of an endoscope, many user causes a normal light image to be displayed on a main monitor and confirms a special light image only in a case where a special observation is required.

### SUMMARY

An endoscopic image processing device according to one aspect of the present disclosure includes one or more processors. The one or more processors are configured to: acquire a first signal related to an image with a first condition; acquire a second signal related to an image with a second condition different from the first condition; create a first image from the first signal; create a second image from the second signal; determine whether visibility of at least a part of the second image is equal to or higher than a second threshold, or lower than the second threshold; and create different notification information depending on whether the visibility is equal to or higher than the second threshold, or lower than the second threshold. The one or more processors create at least one kind of: the notification information for guiding to the second image when the visibility is equal to or higher than the second threshold; or the notification information for notifying that the second image is not suitable for viewing when the visibility is lower than the second threshold.

In a method for operating an endoscopic image processing device according to one aspect of the present disclosure, one or more processors included in the endoscopic image processing device are configured to: acquire a first signal related to an image with a first condition; acquire a second signal related to an image with a second condition different from the first condition; output, to one or more displays, a first image created from the first signal, and a second image created from the second signal; determine whether visibility of a part of the second image is equal to or higher than a second threshold or lower than the second threshold; and create notification information for guiding to the second image when the visibility is equal to or higher than the second threshold, or create notification information for notifying that the second image is not suitable for viewing when the visibility is lower than the second threshold.

### BRIEF DESCRIPTION OF DRAWINGS

FIG. 1 is a view showing an appearance of an endoscope system according to each embodiment of the present disclosure;

FIG. 2 is a block diagram showing a configuration example of an endoscopic image processing device according to each embodiment;

FIG. 3 is a block diagram showing a configuration example of functional sections of the endoscopic image processing device according to each embodiment;

FIG. 4 is a chart showing an example of how a first image and a second image are displayed with respect to a configuration of a display, in each embodiment;

FIG. 5 is a flowchart showing a first example of basic processing by the endoscopic image processing device, in each embodiment;

FIG. 6 is a flowchart showing a second example of the basic processing by the endoscopic image processing device, in each embodiment;

FIG. 7 is a flowchart showing a third example of the basic processing by the endoscopic image processing device, in each embodiment;

FIG. 8 is a flowchart showing a fourth example of the basic processing by the endoscopic image processing device, in each embodiment;

FIG. 9 is a chart showing several examples in which notification information is made different depending on second visibility in the second image, in each embodiment;

FIG. 10 is a flowchart showing specific processing of an endoscopic image processing device in a first embodiment of the present disclosure;

FIG. 11 is a flowchart showing specific processing of an endoscopic image processing device in a second embodiment of the present disclosure;

FIG. 12 is a flowchart showing specific processing of an endoscopic image processing device in a third embodiment of the present disclosure;

FIG. 13 is a flowchart showing specific processing of an endoscopic image processing device in a fourth embodiment of the present disclosure;

FIG. 14 is a chart showing an example of how an image display and a notification are performed on a first display and a second display depending on visibility, in each embodiment;

FIG. 15 is a chart showing an example of how the image display and the notification are performed on one display depending on the visibility, in each embodiment;

FIG. 16 is a chart showing one example of a display change on displays when an endoscopic examination is being performed, in each embodiment;

FIG. 17 is a chart showing another example of the display change on the displays when the endoscopic examination is being performed, in each embodiment;

FIG. 18 is a chart showing a display example of an image depending on the visibility when one image is displayed on the display, in each embodiment; and

FIG. 19 is a chart showing a display example of images depending on the visibility when two images are displayed respectively on a main screen and a sub screen, in each embodiment.

### DESCRIPTION OF EMBODIMENTS

Hereinafter, embodiments of the present invention will be described with reference to drawings. However, the present invention is not limited by the embodiments to be described below.

Note that in the description in the drawings, the same or corresponding elements are attached with the same reference signs as appropriate. In addition, the drawings are schematic, and care should be taken to the fact that a relationship among the lengths of the respective elements, a ratio among the lengths of the respective elements, the number of the respective elements, and the like are sometimes different from the actual ones for simplification of the description. Furthermore, the respective drawings sometimes include parts in which the relationships and ratios among the lengths of the elements are different.

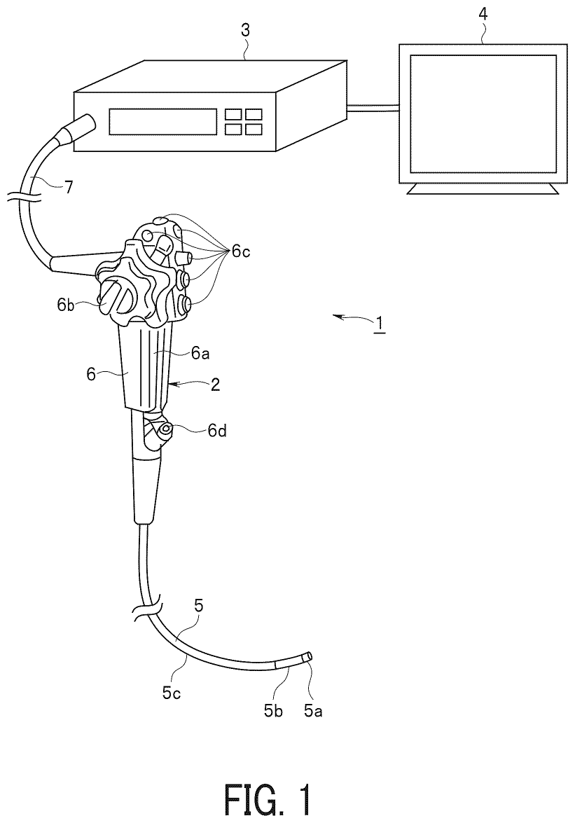

FIG. 1 to FIG. 19 show the respective embodiments of the present disclosure. FIG. 1 is a view showing an appearance of an endoscope system 1 according to each embodiment.

The endoscope system 1 includes an endoscope 2, an endoscopic image processing device 3, and a display 4. The endoscope 2, the endoscopic image processing device 3, and the display 4 are hardware.

The endoscope 2 includes an insertion portion 5, an operation portion 6, and a universal cable 7.

The insertion portion 5 is an elongated part configured to be inserted into a subject. Note that the subject into which the insertion portion 5 is inserted is supposed to be a human body as an example, but not limited to the human body and may be another living body such as an animal, or a non-biological object such as a machine or a structure.

The insertion portion 5 includes, in the following order from the distal end side toward the proximal end side, a distal-end constituting portion 5a, a bending portion 5b, and a flexible tube portion 5c.

The endoscope 2 is configured as an electronic endoscope and includes an image pickup system in the distal-end constituting portion 5a. The image pickup system includes an objective lens that forms an optical image of a subject, and an image pickup device that photoelectrically converts the optical image formed by the objective lens and outputs an electric signal. The image pickup device creates image signals in frame units and transmits the created image signals to the endoscopic image processing device 3.

Note that the image pickup device is not limited to being provided in the distal-end constituting portion 5a of the insertion portion 5. For example, a configuration may be employed in which a relay optical system is provided in the insertion portion 5 and the operation portion 6, and a camera head is attached to the operation portion 6. If such a configuration is employed, the optical image formed by the objective lens is transmitted by the relay optical system and picked up by the image pickup device in the camera head.

The bending portion 5b is a part configured to be bendable in two directions, i.e., up and down directions, or in four directions, i.e., up, down, left, and right directions, for example. The bending portion 5b is disposed on the proximal end side of the distal-end constituting portion 5a. When the bending portion 5b is bent, the direction of the distal-end constituting portion 5a is changed, and an irradiation direction of illumination light and an observation direction of the image pickup system are changed. In addition, the bending portion 5b is bent also for improving the insertion performance of the insertion portion 5 in the subject.

The flexible tube portion 5c is a tubular member having a flexibility. The flexible tube portion 5c is provided on the proximal end side of the bending portion 5b. Note that description is made here by taking a case where the endoscope 2 is a flexible endoscope having the flexible tube portion 5c, as an example. However, the endoscope 2 may be a rigid endoscope having a configuration in which a part corresponding to the flexible tube portion 5c is rigid. In addition, the endoscope 2 may be any type of the following: a type in which entirety of the endoscope 2 is disposal; a type in which the entirety is reused after reprocessing; or a type in which a part of the endoscope 2 is disposable.

The operation portion 6 is disposed on the proximal end side of the insertion portion 5. The operation portion 6 includes a grasping portion 6a, a bending operation knob 6b, operation buttons 6c, and a treatment instrument insertion port 6d.

The grasping portion 6a is a part for a user to grasp the endoscope 2 with the palm.

The bending operation knob 6b is an operation device for operating bending of the bending portion 5b. The bending operation knob 6b is operated by using, for example, the thumb of the hand grasping the grasping portion 6a. The bending operation knob 6b is connected to the bending portion 5b with bending wires. When the bending operation knob 6b is operated, the bending wires are pulled, to thereby cause the bending portion 5b to bend.

The operation buttons 6c include a plurality of buttons for operating the endoscope 2. Some examples of the operation buttons 6c include a gas/liquid feeding button, a suction button, and a button related to image pickup.

The treatment instrument insertion port 6d is a proximal end side opening of a treatment instrument channel disposed in the insertion portion 5 and the operation portion 6. When a treatment instrument is inserted from the treatment instrument insertion port 6d, the distal end of the treatment instrument protrudes from a distal end side opening of the treatment instrument channel, the distal end side opening being formed at the distal-end constituting portion 5a. In this state, various kinds of treatment are performed on the subject with the treatment instrument.

The universal cable 7 is extended, for example, from a side surface on the proximal end side of the operation portion 6, and connected to the endoscopic image processing device 3.

The endoscopic image processing device 3 receives the image signals in frame units from the image pickup device. The endoscopic image processing device 3 performs image processing on the acquired image signals and outputs the image signals after the processing to the display 4. In addition, the endoscopic image processing device 3 serves also as an endoscope control device configured to control the endoscope 2.

The endoscopic image processing device 3 may serve also as an illumination device the emits illumination light. Alternatively, the illumination device may be provided separately from the endoscopic image processing device 3. The illumination device is capable of emitting, for example, normal light such as white light and special light whose spectral distribution is different from that of the normal light. As will be described later, the endoscopic image processing device 3 may include a computer-aided detection (CADe) function, or a computer-aided diagnosis (CADx) function, or the CADe or the CADx may be mounted in another processor which is configured to be capable of communicating with the endoscopic image processing device 3.

The display 4 is a display device (display section) configured to receive an image signal and display an endoscopic image. Note that the display 4 is not required to have a configuration unique to the endoscope system 1. For example, the display 4 provided separately from the endoscope system 1 may be used by connecting the display 4 to the endoscopic image processing device 3. In addition, as will be described later, the number of the display 4 is not limited to one, but may be more than one.

The endoscope system 1 may include a sound generation device such as a speaker or buzzer, which generates sound or voice, integrally with or separately from the endoscopic image processing device 3 or the display 4. Furthermore, the endoscope system 1 may include a vibration device that generates vibration integrally with or separately from or the display 4.

The display 4 is an example of a notification device (notification section) to which notification information as visual information is outputted. The sound generation device is an example of a notification device to which notification information as sound information or voice information is outputted. The vibration device is an example of a notification device to which notification information as vibration information is outputted. In other words, the examples of the notification information include one or more of the visual information, the sound information, the voice information, or the vibration information.

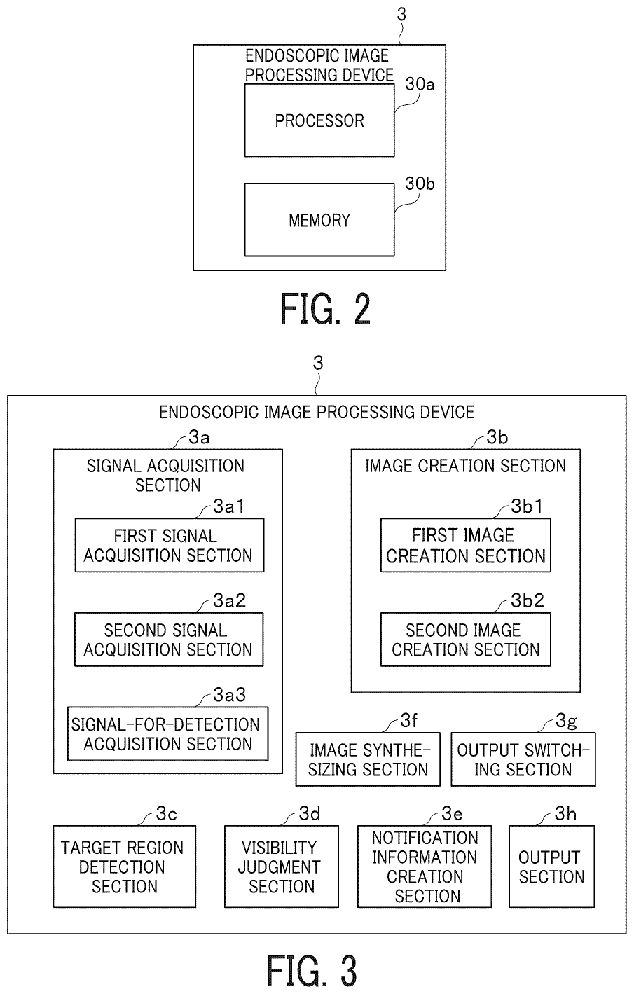

FIG. 2 is a block diagram showing a configuration example of the endoscopic image processing device 3 according to each embodiment.

Respective functional sections (see FIG. 3) of the endoscopic image processing device 3 may be configured by an electronic circuit. All or some of the functional sections of the endoscopic image processing device 3 may be configured by a processor 30a and a memory 30b as shown in FIG. 2. The processor 30a and the memory 30b are hardware. The processor 30a is configured by an application specific integrated circuit (ASIC) including a central processing unit (CPU), etc., a field programmable gate array (FPGA), or the like. The memory 30b is a non-volatile storage medium and stores a computer program that causes the processor 30a to operate as the respective functional sections. The processor 30a reads and executes the computer program stored in the memory 30b, to operate as the respective functional sections in the endoscopic image processing device 3. The endoscopic image processing device 3 may include a plurality of processors 30a.

FIG. 3 is a block diagram showing a configuration example of the functional sections of the endoscopic image processing device 3 according to each embodiment. Note that FIG. 3 enumerates the functional sections according to each embodiment, but as needed, some of the functional sections may be omitted or another functional section may be added. Note that the respective components are not required to be mounted in one device, but components mounted in another device may be connected via a communication device to be used as the components constituting the endoscopic image processing device 3.

The endoscopic image processing device 3 includes a signal acquisition section 3a, an image creation section 3b, a target region detection section 3c, a visibility judgment section 3d, a notification information creation section 3e, an image synthesizing section 3f, an output switching section 3g, and an output section 3h.

The signal acquisition section 3a includes a first signal acquisition section 3a1, a second signal acquisition section 3a2, and a signal-for-detection acquisition section 3a3.

The first signal acquisition section 3al acquires a first signal related to an image with a first condition. One example of the first condition includes a condition for acquiring the first signal by illuminating a subject with normal light (white light, etc.). The first condition may include various setting conditions related to photographing (light emission intensity of the normal light, an exposure time, a signal amplification factor, etc.). The first condition may further include a condition for first image processing to be performed on the first signal for generating a normal light image.

The second signal acquisition section 3a2 acquires a second signal related to an image with a second condition different from the first condition. One example of the second condition includes a condition for acquiring the second signal by illuminating the subject with special light. The second condition may include various setting conditions related to photographing (light emission intensity of the special light, an exposure time, a signal amplification factor, presence or absence and a type of an optical filter, etc.). The second condition may further include a condition for second image processing (image processing different from the first image processing) to be performed on the second signal for generating a special light image. Furthermore, the second condition is not limited to illuminating the special light. For example, the second condition may be a condition under which special image processing (image processing different from the first image processing and the second image processing) is performed on the first signal obtained by irradiating the subject with the normal light and an image corresponding to the special light image is obtained.

The signal-for-detection acquisition section 3a3 acquires a signal for detection. The signal for detection is a signal to be used for an artificial intelligence (AI), which will be described later, of the target region detection section 3c to detect a target region such as a lesion candidate region. The signal-for-detection acquisition section 3a3 and the second signal acquisition section 3a2 may be integrated. In this case, the second signal serves also as the signal for detection. Alternatively, the signal-for-detection acquisition section 3a3 and the first signal acquisition section 3al may be integrated. In this case, the first signal serves also as the signal for detection.

The image creation section 3b includes a first image creation section 3b1 and a second image creation section 3b2. The first image creation section 3b1 performs the first image processing on the first signal to create a first image. The first image (normal light image) created from the first signal acquired by illuminating the subject with the normal light can be used as a normal observation image.

The second image creation section 3b2 performs the second image processing on the second signal to create a second image. The second image (special light image) created from the second signal acquired by illuminating the subject with the special light can be used as a recognition image for detecting a target region.

The target region detection section 3c detects the target region (at least a part of the image) based on the signal for detection. One example of the target region is a lesion candidate region, which is a candidate for a lesion region (note that it may be a lesion region when the lesion candidate region is determined as the lesion region). However, the target region is not limited to the lesion candidate region but may be a normal region. In a case where detection of the lesion candidate region is not required, for example, an obesity treatment, a nasal mucosa cauterization, an endoscopic diverticulum septum incision, a normal region may be detected as a target region. Examples of the target region as the normal region include a fat, a blood vessel, a bleeding point, a nerve, a ureter, a urethra, or the like.

If the second signal serves also as the signal for detection, the target region detection section 3c detects the target region from the second image created from the second signal. The target region detected from the signal for detection or the second image may be used as a target region for the first image. Alternatively, the target region detection section 3c may further detect a target region from the first image.

The target region detection section 3c detects the target region using the AI, for example. In this case, the target region detection section 3c includes an AI for target region detection. When the AI detects the target region in the first image, the AI may further detect a first score indicating an accuracy of the target region in the first image. When the AI detects the target region in the second image, the AI may further detect a second score indicating an accuracy of the target region in the second image. The number of detectors (discriminators) included in the target region detection section 3c is not limited, as long as the number is one or more. If the target region detection section 3c includes one detector, the one detector detects the target region in the first image and the target region in the second image. If the target region detection section 3c includes two or more detectors, a first detector may detect the target region in the first image and a second detector may detect the target region in the second image.

The visibility judgment section 3d determines whether visibility (second visibility) of at least a part of the second image (specifically, the target region in the second image) is equal to or higher than a second threshold (a certain threshold), or lower than the second threshold. Furthermore, in addition to the second image, the visibility judgment section 3d may determine whether first visibility of at least a part of the first image (target region) is equal to or higher than a first threshold, or lower than the first threshold. Here, the first threshold is a threshold for the first visibility of the target region in the first image. The second threshold is a threshold for the second visibility of the target region in the second image.

If the first visibility is equal to or higher than the first threshold, the visibility judgment section 3d determines that the first visibility is high. On the other hand, if the first visibility is lower than the first threshold, the visibility judgment section 3d determines that the first visibility is low. If the second visibility is equal to or higher than the second threshold, the visibility judgment section 3d determines that the second visibility is high. On the other hand, if the second visibility is lower than the second threshold, the visibility judgment section 3d determines that the second visibility is low.

Therefore, hereinafter, the case where “the first visibility is high” indicates that “the first visibility is equal to or higher than the first threshold”, and the case where “the first visibility is low” indicates that “the first visibility is lower than the first threshold”. Similarly, the case where “the second visibility is high” indicates that “the second visibility is equal to or higher than the second threshold”, and the case where “the second visibility is low” indicates that “the second visibility is lower than the second threshold”.

The visibility judgment section 3d may include a second AI specialized for visibility estimation. The second AI estimates a visibility score of the target region, for example. The visibility judgment section 3d compares the estimated visibility score with the threshold (the first threshold or the second threshold corresponding to the visibility score), thereby judging the visibility of the target region.

The notification information creation section 3e creates different notification information depending on whether the visibility (second visibility) of at least a part of the second image (specifically, the target region) is equal to or higher than the second threshold (certain threshold), or the visibility is lower than the second threshold. The notification information is information to be notified to a user, and information for urging caution, for example. In addition, “creating different notification information” includes creating the notification information and not creating notification information (creating null (zero) notification information).

The notification information creation section 3e may create the notification information depending not only on the level of the second visibility but also further on the level of the first visibility. For example, in the case where the first visibility is lower than the first threshold, the notification information creation section 3e may create different notification information depending on whether the second visibility is equal to or higher than the second threshold, or the second visibility is lower than the second threshold. Furthermore, the notification information creation section 3e may create an icon indicating the position of the target region.

When the AI creates the score indicating the accuracy of the target region or when the second AI creates the visibility score, the notification information creation section 3e may create the notification information that indicates the score indicating the accuracy or the visibility score.

The image synthesizing section 3f synthesizes the first image and the second image to create a synthetic image. The image synthesizing section 3f creates the synthetic image, when the first visibility in the first image is lower than the first threshold and the second visibility in the second image is equal to or higher than the second threshold, for example. Therefore, when the first visibility is equal to or higher than the first threshold, or the second visibility is lower than the second threshold, the image synthesizing section 3f does not have to create the synthetic image (however, may create the synthetic image as needed).

The image synthesizing section 3f may synthesize the first image and the second image at the ratio of 1 to 1, or by setting a synthetic ratio. The synthetic ratio is set based on the visibility, for example. Furthermore, the image synthesizing section 3f may create the synthetic image by synthesizing only the target region in the second image with the first image (or by synthesizing the target region in the second image after setting the synthetic ratio thereof based on the visibility).

In the case of displaying one image on one display 4, for example, the output switching section 3g performs switching between the first image and the second image, to cause the output section 3h to output the switched image along with the notification information. In addition, when the image synthesizing section 3f creates the synthetic image, the output switching section 3g performs switching among the first image, the second image, and the synthetic image, to cause the output section 3h to output one or more of these images, along with the notification information. However, if switching of the images is not required, there is no need to provide the output switching section 3g.

The output section 3h outputs the image based on at least one of the first image or the second image to the display 4 and outputs the notification information to the notification device (notification section). When the image synthesizing section 3f creates the synthetic image, the output section 3h may output the synthetic image to the display 4. Therefore, examples of the image based on at least one of the first image or the second image include the first image itself, the second image itself, the synthetic image, etc.

In addition, the output section 3h may output, to the display 4, the icon indicating the position of the target region, which has been created by the notification information creation section 3e, along with the first image (and/or the second image (may be synthetic image)). Thus, the endoscope system 1 can function as the CADe.

Here, description will be made on several examples of the visibility determination by the visibility judgment section 3d. Note that calculation of the visibility, which will be described below, is not limited to a case of calculating only one kind of visibility. A plurality of kinds of visibility may be calculated, and based on the result of the calculation, comprehensive visibility may be calculated. In addition, the first threshold and the second threshold are set respectively to the values corresponding to the determination methods of the visibility.

The visibility judgment section 3d may determine the first visibility based on, for example, information on the target region and information on a region outside the target region (peripheral region) in the first image. Similarly, the visibility judgment section 3d may determine the second visibility based on, for example, information on the target region and information on a region outside the target region (peripheral region) in the second image.

For example, the visibility judgment section 3d calculates a first color difference between the target region and the region outside the target region in the first image. Then, the visibility judgment section 3d compares the first color difference with the first threshold by using the first color difference as the first visibility, thereby judging the first visibility.

Furthermore, the visibility judgment section 3d calculates a second color difference between the target region and the region outside the target region in the second image. Then, the visibility judgment section 3d compares the second color difference with the second threshold by using the second color difference as the second visibility, thereby judging the second visibility. Here, the color difference is defined as a Euclidean distance in a color space, for example.

The visibility judgment section 3d detects a blood vessel from the target region in the first image, and detects a blood vessel from the region outside the target region in the first image. The visibility judgment section 3d calculates a first blood vessel amount difference between the target region and the region outside the target region in the first image, and compares the first blood vessel amount difference with the first threshold by using the first blood vessel amount difference as the first visibility.

The visibility judgment section 3d detects a blood vessel from the target region in the second image, and detects a blood vessel from the region outside the target region in the second image. The visibility judgment section 3d calculates a second blood vessel amount difference between the target region and the region outside the target region in the second image, and compares the second blood vessel amount difference with the second threshold by using the second blood vessel amount difference as the second visibility.

For example, the visibility judgment section 3d calculates, for the first image or the second image, a difference between a ratio of the area of the blood vessel detected in the target region and a ratio of the area of the blood vessel detected in the region outside the target region, as the first blood vessel amount difference or the second blood vessel amount difference.

Note that, instead of using the ratio of the area of the blood vessel as the blood vessel amount, an average of sums, each of which is a sum of the areas of the blood vessels included in a unit area in the target region or the region outside the target region, may be used as the blood vessel amount. In addition, instead of the average of the sums, each of which is the sum of the areas of the blood vessels included in the unit area, an average of sums, each of which is a sum of the lengths of the blood vessels included in the unit area, may be used as the blood vessel amount.

If the target region is a lesion region such as a tumor, there is a case where more blood vessels are detected in the target region than in the region outside the target region. Therefore, such a determination method is effective for the case of detecting a lesion candidate region such as a tumor candidate.

Furthermore, for example, when the AI detects the target region in the first image, the target region in the second image, and the first and second scores, the visibility judgment section 3d may compare the first score with the first threshold by using the first score as the first visibility, thereby judging the first visibility, and compare the second score with the second threshold by using the second score as the second visibility, thereby judging the second visibility. If the target region is the lesion candidate region, for example, the AI detects the first score and the second score as lesion scores, respectively.

For example, the visibility judgment section 3d extracts a first edge from the target region in the first image, to calculate a first edge amount. The visibility judgment section 3d compares the first edge amount with the first threshold by using the first edge amount as the first visibility, thereby judging the first visibility. The visibility judgment section 3d extracts a second edge from the target region in the second image, to calculate a second edge amount. The visibility judgment section 3d compares the second edge amount with the second threshold by using the second edge amount as the second visibility, thereby judging the second visibility. Here, the edge amount may be calculated based on the total amount of the edges detected in the target region, for example. If the edge amount is equal to or greater than the threshold, the target region can be clearly distinguished from regions other than the target region, which leads to a determination that the visibility is high.

When the second AI estimates the visibility score as described above, the visibility judgment section 3d may determine that the visibility of the target region is high if the visibility score of the target region is equal to or higher than the threshold, and may determine that the visibility of the target region is low if the visibility score of the target region is lower than the threshold.

For example, the visibility judgment section 3d may judge the visibility depending on whether the target region has been detected. If the target region is detected from the first image, the visibility judgment section 3d may determine that the first visibility is equal to or higher than the first threshold. On the other hand, if no target region is detected from the first image, the visibility judgment section 3d may determine that the first visibility is lower than the first threshold. If the target region is detected from the second image, the visibility judgment section 3d may determine that the second visibility is equal to or higher than the second threshold. On the other hand, if no target region is detected from the second image, the visibility judgment section 3d may determine that the second visibility is lower than the second threshold.

For example, the visibility judgment section 3d may judge the visibility based on a matching degree of the positions of the target regions. Specifically, if the target region is detected in the same position in the first and second images (the matching degree of the positions of the target regions is high), the visibility judgment section 3d determines that the first visibility of the target region in the first image is high and also the second visibility of the target region in the second image is high. In addition, if the target region is detected in only one of the same positions in the first and second images, the visibility judgment section 3d determines that the visibility of the detected target region in one of the first and second images is high, and the visibility of the target region in the other in which the target region has not been detected is low.

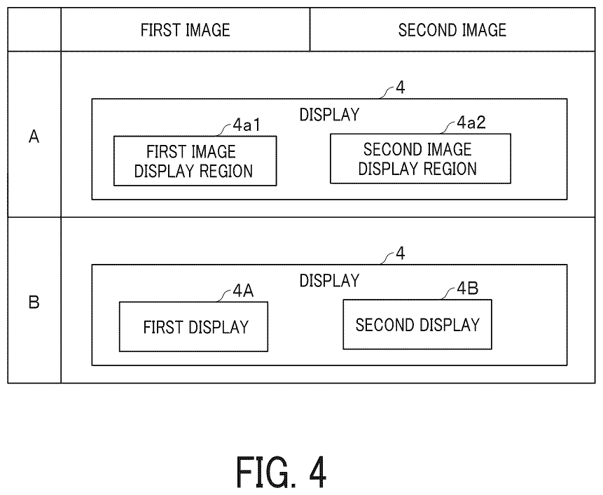

FIG. 4 is a chart showing an example of how the first image and the second image are displayed with respect to a configuration of the display 4, in each embodiment.

The section A in FIG. 4 shows an example in which the display 4 is configured as one display device. The display 4 includes, in a part of a display screen thereof, a first image display region 4al for displaying the first image, and includes, in another part of the display screen thereof, a second image display region 4a2 for displaying the second image. In this case, for example, the output section 3h may output the first image and the second image to the display 4, with the first and second images arranged in parallel.

Note that the first image display region 4al may be set as a region larger than the second image display region 4a2. In this case, the output section 3h may create a reduced second image, and may output the first image and the reduced second image to the display 4, with the first image and the reduced second image arranged in parallel.

In addition, the first image display region 4al and the second image display region 4a2 do not have to be provided individually, but the second image display region 4a2 may be arranged inside the first image display region 4a1, for example. In this case, the output section 3h may create the reduced second image, and may output the first image and the reduced second image to the display 4, with the reduced second image superimposed on a part not overlapping the target region in the first image.

The section B in FIG. 4 shows an example in which the display 4 includes two display devices, i.e., a first display 4A and a second display 4B. In this example, the first image is displayed on the first display 4A, and the second image is displayed on the second display 4B.

Note that the first display 4A and the second display 4B may have different screen sizes. When the screen sizes are different, the screen size of the first display 4A may be larger than the screen size of the second display 4B. This is because the first image, which is the normal light image, is a main image mainly observed by a user. In this case, the first display 4A serves as a main display and the second display 4B serves as a sub display.

Note that, hereinafter, as appropriate, the first image display region 4a1 or the screen of the first display 4A is referred to as a main screen, and the second image display region 4a2 or the screen of the second display 4B is referred to as a sub screen.

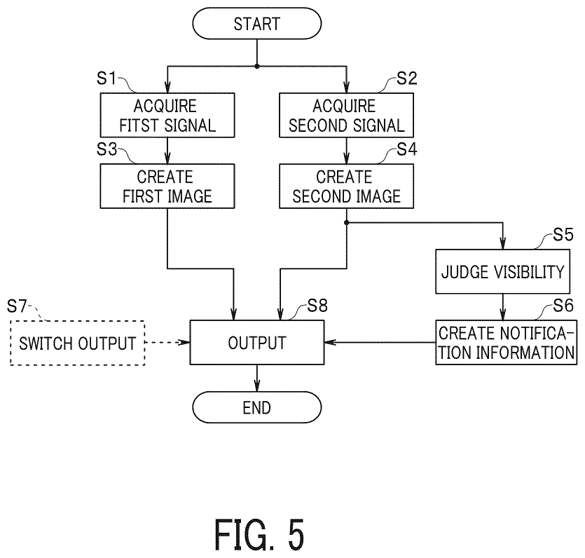

FIG. 5 is a flowchart showing a first example of basic processing by the endoscopic image processing device 3, in each embodiment.

When the processing starts, the first signal acquisition section 3al acquires the first signal (Step S1), and the second signal acquisition section 3a2 acquires the second signal (Step S2).

The first image creation section 3b1 creates the first image from the first signal acquired by the first signal acquisition section 3al (Step S3).

The second image creation section 3b2 creates the second image from the second signal acquired by the second signal acquisition section 3a2 (Step S4).

The visibility judgment section 3d determines whether the second visibility of the target region in the second image is equal to or higher than the second threshold, or lower than the second threshold (Step S5).

The notification information creation section 3e creates different notification information depending on whether the second visibility of the target region in the second image is equal to or higher than the second threshold, or lower than the second threshold (Step S6). Examples of the notification information will be described later with reference to FIG. 9.

The output section 3h outputs the image based on at least one of the first image or the second image to the display 4, and outputs the notification information to the notification device (Step S8).

At this time, if the display 4 is configured by one display device, the output switching section 3g may switch which of the first image and the second image is outputted by the output section 3h to the display 4 (Step S7).

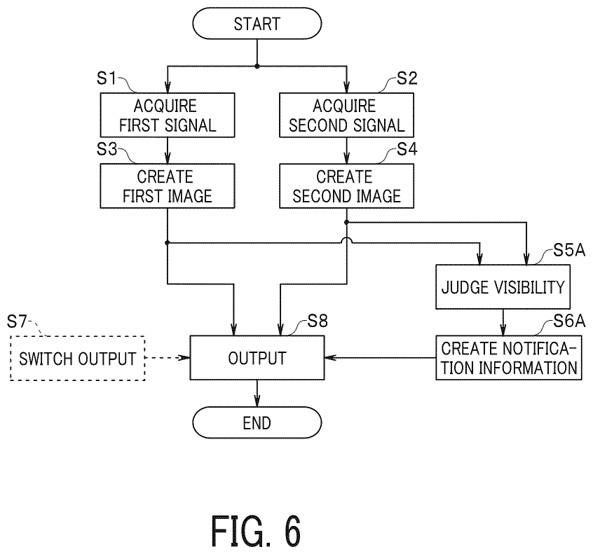

After Step S8 is performed, this processing ends. Note that, when a next frame image is outputted, the processing shown in FIG. 5 is performed again (the same is true for the processing in FIG. 6 or subsequent processing).

FIG. 6 is a flowchart showing a second example of the basic processing by the endoscopic image processing device 3, in each embodiment.

When the processing starts, the processing in the above-described Steps S1 to S4 is performed.

The visibility judgment section 3d determines whether the second visibility of the target region in the second image is equal to or higher than the second threshold, or lower than the second threshold. Furthermore, the visibility judgment section 3d determines whether the first visibility of the target region in the first image is equal to or higher than the first threshold, or lower than the first threshold (Step S5A).

The notification information creation section 3e creates different notification information depending on: the case where the first visibility is lower than the first threshold and the second visibility is equal to or higher than the second threshold; or the case where the first visibility is lower than the first threshold and the second visibility is lower than the second threshold (Step S6A).

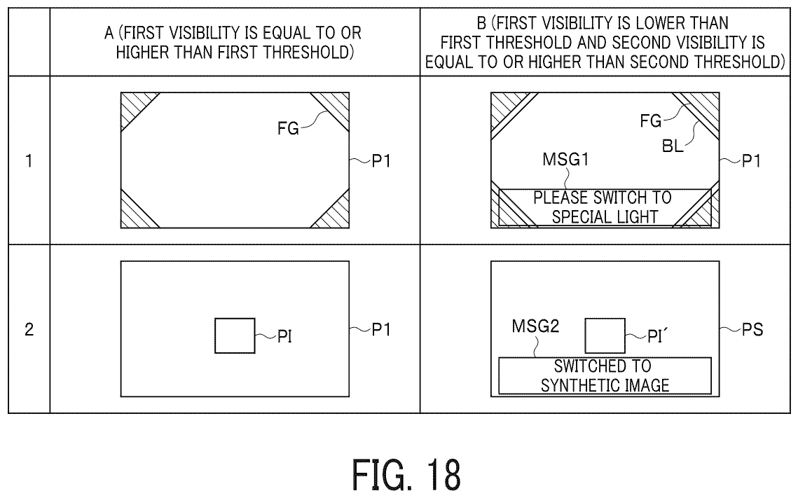

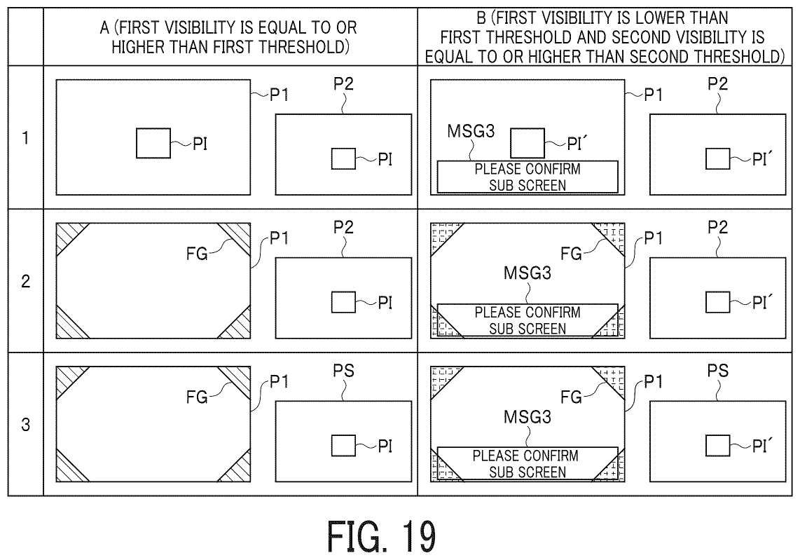

The example of the notification information depending on the second visibility in the case where the first visibility is lower than the first threshold will be described later with reference to FIG. 9, similarly as described above. Note that, if the first visibility is equal to or higher than the first threshold, it is not necessary for the user to move his/her line of sight to the second image, or the like. Therefore, the notification information creation section 3e does not have to create the notification information or may create notification information for notifying that the observation of the first image may be continued (see FIG. 18 and FIG. 19).

Then, after Step S8 (further Step S7 as needed) is performed, this processing ends.

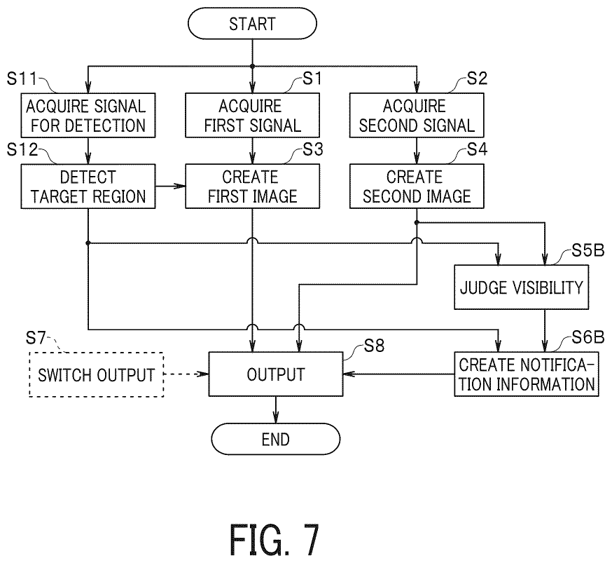

FIG. 7 is a flowchart showing a third example of the basic processing by the endoscopic image processing device 3, in each embodiment.

When the processing starts, the processing in the above-described Step S1 to Step S4 is performed.

Furthermore, the signal-for-detection acquisition section 3a3 acquires the signal for detection (Step S11).

The target region detection section 3c uses the AI (discriminator), for example, to detect the target region based on the signal for detection acquired by the signal-for-detection acquisition section 3a3 (Step S12). As described above, the signal for detection may be the same as either the first signal or the second signal, or may be different from the first signal and the second signal. The detection result of the target region detection section 3c is transmitted to the first image creation section 3b1.

The visibility judgment section 3d may be configured to receive the detection result from the target region detection section 3c. In this case, it may be determined whether the second visibility of the detected target region in the second image is equal to or higher than the second threshold, or lower than the second threshold (Step S5B).

The notification information creation section 3e creates different notification information depending on whether the second visibility is equal to or higher than the second threshold, or lower than the second threshold (Step S6B) (see FIG. 9, etc.). Note that, when creating the icon indicating the position of the target region, the notification information creation section 3e may create the icon having a different shape depending on whether the second visibility is equal to or higher than the second threshold, or the second visibility is lower than the second threshold (display control may be performed).

The icon indicating the position of the target region created by the notification information creation section 3e is, in the processing in Step S8, outputted, by the output section 3h, to the first display 4A which serves also as the notification device, for example, and displayed on the first image on the first display 4A.

After Step S8 (further Step S7 as needed) is thus performed, this processing ends.

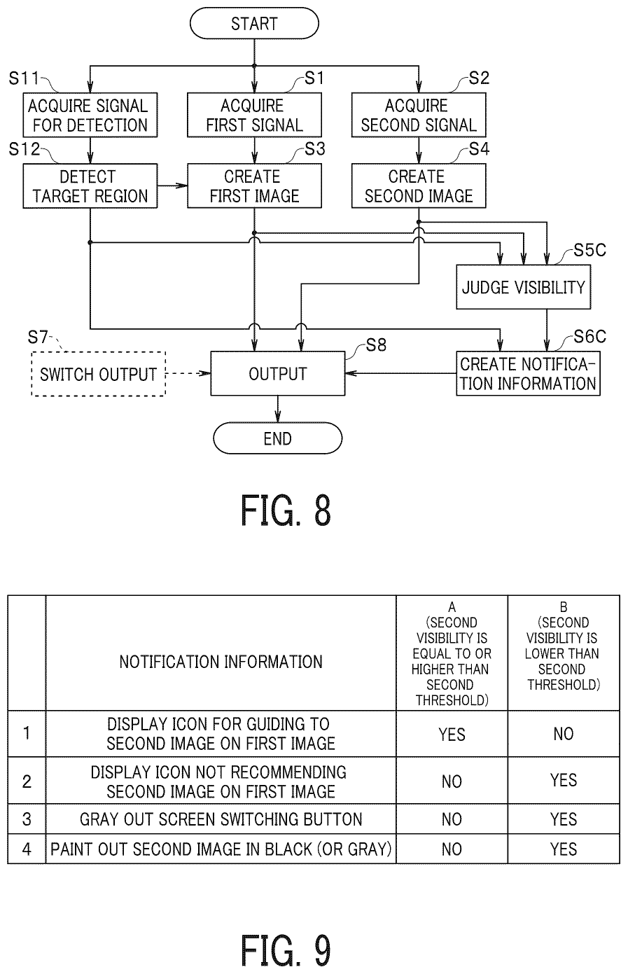

FIG. 8 is a flowchart showing a fourth example of the basic processing by the endoscopic image processing device 3, in each embodiment.

When the processing starts, the processing in the above-described Steps S1 to S4 and S11 to S12 is performed.

The visibility judgment section 3d determines whether the second visibility in the second image of the target region detected by the target region detection section 3c is equal to or higher than the second threshold, or lower than the second threshold. Further, the visibility judgment section 3d determines whether the first visibility in the first image of the target region detected by the target region detection section 3c is equal to or higher than the first threshold, or lower than the first threshold (Step S5C).

The notification information creation section 3e creates different notification information depending on: the case where the first visibility is lower than the first threshold and the second visibility is equal to or higher than the second threshold; or the case where the first visibility is lower than the first threshold and the second visibility is lower than the second threshold (Step S6C) (see FIG. 9, etc.).

Then, after Step S8 (further Step S7 as needed) is performed, this processing ends.

FIG. 9 is a chart showing several examples in which the notification information is made different depending on the second visibility in the second image, in each embodiment. FIG. 9 shows an example of a case where the notification information is the visual information.

The section 1 in FIG. 9 shows an example of the notification information in the case where the first image and the second image are displayed simultaneously on the main screen and the sub screen, respectively. If the second visibility is equal to or higher than the second threshold, an icon for guiding to the second image is displayed on the first image (the section 1A in FIG. 9). If the second visibility is lower than the second threshold, the icon for guiding to the second image is not displayed on the first image (the section 1B in FIG. 9).

The section 2 in FIG. 9 shows another example of the notification information in the case where the first image and the second image are displayed simultaneously on the main screen and the sub screen, respectively. If the second visibility is equal to or higher than the second threshold, an icon indicating that the user is not recommended to view the second image is not displayed on the first image (the section 2A in FIG. 9). If the second visibility is lower than the second threshold, the icon indicating that the user is not recommended to view the second image is displayed on the first image (the section 2B in FIG. 9). The icon indicating that the user is not recommended to view the second image is an example of the notification information for notifying that the second image is not suitable for viewing.

The section 3 in FIG. 9 shows an example of the notification information in the case where the first image and the second image are switched to be displayed on one screen. In this case, a screen switching button is displayed with either the first image or the second image on the display 4. The screen switching button is operated by using an input device provided in the endoscopic image processing device 3 or connected to the endoscopic image processing device 3. Examples of the input device connected to the endoscopic image processing device 3 include a touch panel provided to the display 4, a keyboard, and a mouse.

In general, if the screen switching button is operated while the first image is being displayed, the second image is displayed in place of the first image. If the screen switching button is operated while the second image is being displayed, the first image is displayed in place of the second image.

If the second visibility is equal to or higher than the second threshold, the screen switching button is not grayed out (the section 3A in FIG. 9). Therefore, the image to be displayed on the display 4 can be switched from the first image to the second image, and can be switched from the second image to the first image.

If the second visibility is lower than the second threshold, the screen switching button at the time when the first image is being displayed is grayed out (the section 3B in FIG. 9). In this case, the image to be displayed on the display 4 cannot be switched from the first image to the second image. Accordingly, the image to be displayed on the display 4 is only the first image (the second image is not displayed).

The section 4 in FIG. 9 shows a yet another example of the notification information in the case where the first image and the second image are simultaneously displayed on the main screen and the sub screen, respectively. If the second visibility is equal to or higher than the second threshold, the second image is not painted out in black (or gray) (the section 4A in FIG. 9). Thus, the user can observe the second image as desired. If the second visibility is lower than the second threshold, the second image is painted out in black (or gray) (the section 4B in FIG. 9). Thus, the second image is automatically excluded from an object to be observed by the user.

### First Embodiment

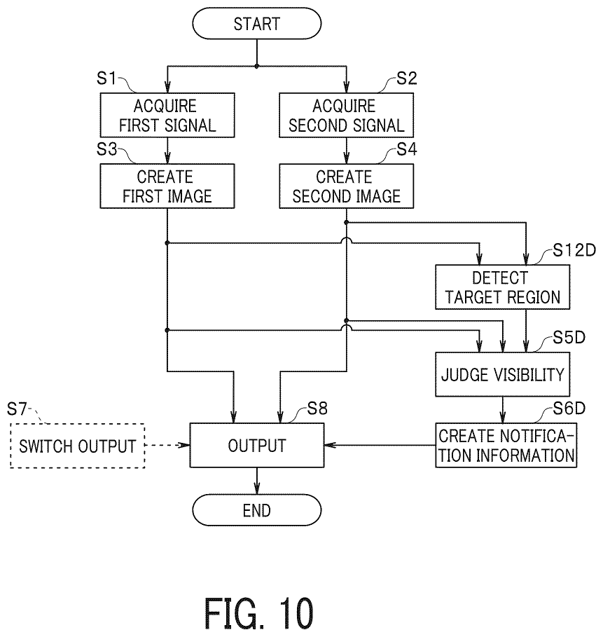

FIG. 10 is a flowchart showing specific processing of an endoscopic image processing device 3 in the first embodiment.

When the processing starts, the processing in the above-described Steps S1 to S4 is performed.

The target region detection section 3c uses the AI, for example, to detect the target region from the first image created by the first image creation section 3b1. Furthermore, the target region detection section 3c uses the AI, for example, to detect the target region from the second image created by the second image creation section 3b2 (Step S12D). As described above, the AI may further detect the lesion scores (the first score and the second score), if the target region is the lesion candidate region.

The visibility judgment section 3d determines whether the first visibility of the target region in the first image is equal to or higher than the first threshold, or lower than the first threshold. Furthermore, the visibility judgment section 3d determines whether the second visibility of the target region in the second image is equal to or higher than the second threshold, or lower than the second threshold (Step S5D). Here, the visibility judgment section 3d calculates the first visibility and the second visibility, based on at least one of the color difference, the edge amount, or the blood vessel amount, as described above, for example.

The notification information creation section 3e creates the notification information for urging caution only when the first visibility is lower than the first threshold and the second visibility is equal to or higher than the second threshold (Step S6D).

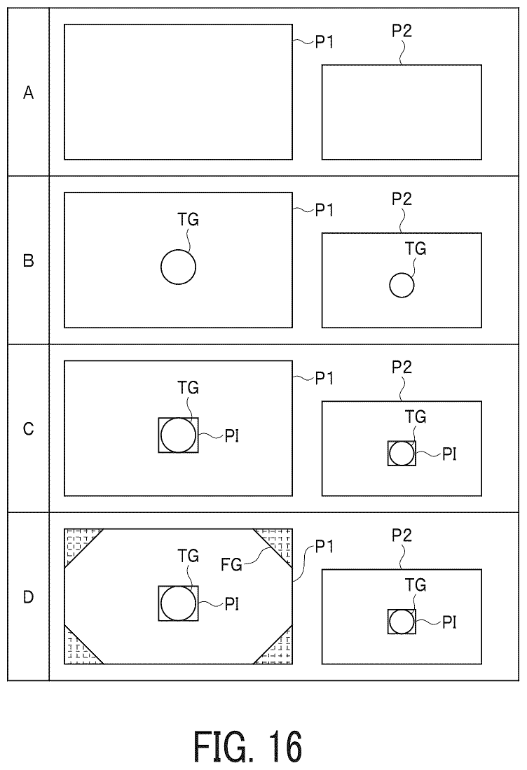

In the processing in Step S8, the output section 3h outputs the image based on at least one of the first image or the second image to the display 4, and outputs the notification information to the notification device. By outputting the notification information for urging caution, for example, at least one of the following is performed: notification by displaying a flag (see the flag FG shown in each of FIG. 16, FIG. 18, and FIG. 19); notification by at least one of a sound, a voice, or characters (see the messages MSG1 to MSG3 shown in FIG. 18 and FIG. 19); or notification in the case where the lesion score detected by the AI exceeds the threshold.

After Step S8 (further, Step S7 as needed) is thus performed, this processing ends.

### Second Embodiment

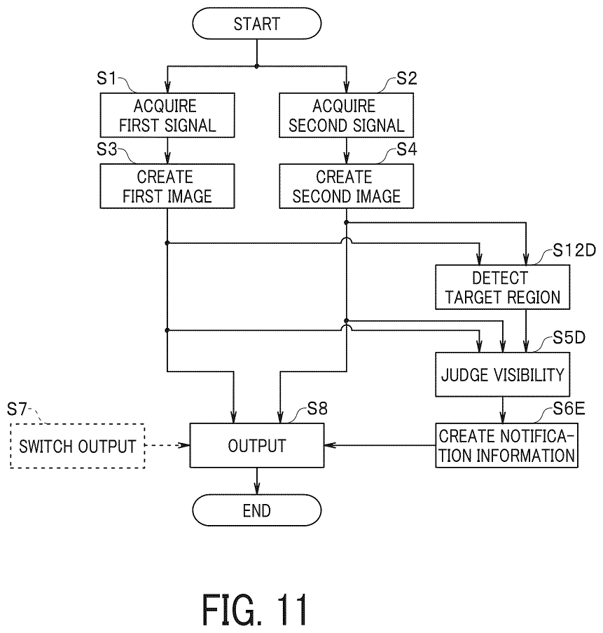

FIG. 11 is a flowchart showing specific processing of an endoscopic image processing device 3 in the second embodiment.

When the processing starts, the processing in the above-described Steps S1 to S4, S12D, and S5D is performed.

The notification information creation section 3e creates the notification information for urging caution only when the first visibility is lower than the first threshold and the second visibility is equal to or higher than the second threshold (Step S6E).

For example, the notification information creation section 3e performs at least one of the following: creating a marker indicating the position of the target region detected from the first image (position information as one of various kinds of notification information), to superimpose the created marker at least on the first image; or creating a maker indicating the position of the target region detected from the second image, to superimpose the created marker at least on the second image. In the former case, the notification information creation section 3e may further superimpose the marker on the second image, while in the latter case, may further superimpose the maker on the first image.

Alternatively, the notification information creation section 3e performs at least one of the following: creating a flag (see the flag FG shown in each of FIG. 16, FIG. 18, and FIG. 19) which is the notification information indicating that the target region has been detected from the first image, to superimpose the created flag at least on the first image; or creating a flag indicating that the target region has been detected from the second image, to superimpose the created flag at least on the second image. In the former case, the notification information creation section 3e may further superimpose the flag on the second image, while in the latter case, may further superimpose the flag on the first image. Note that either the marker or flag, or both the marker and flag may be created.

Furthermore, for example, the notification information creation section 3e further sets a third threshold lower than the first threshold, and sets at least one of a color of the marker or a color of the flag depending on whether the first visibility is lower than the third threshold, the first visibility is equal to or higher than the third threshold and lower than the first threshold, or the first visibility is equal to or higher than the first threshold (see FIG. 16, for example).

Note that the notification information creation section 3e may also classify the second visibility into three cases, and set at least one of the color of the marker or the color of the flag depending on the result of the classification. Furthermore, the cases of the classification of the first visibility and the second visibility by the notification information creation section 3e is not limited to two or three, but may be four or more.

If the second visibility is determined to be equal to or higher than the second threshold, the notification information creation section 3e may further create notification information for notifying a second condition.

In the processing in Step S8, the output section 3h outputs, to the main screen, the first image on which at least one of the marker or the flag, the color of which has been set, is superimposed, and outputs, to the sub screen, the second image (on which at least one of the marker or the flag, the color of which has been set as needed, is superimposed). In addition, the output section 3h may output, to the display 4, the image in which the reduced second image is superimposed on a part not overlapping the target region in the first image.

Thus, after Step S8 (further Step S7 as needed) is performed, this processing ends.

### Third Embodiment

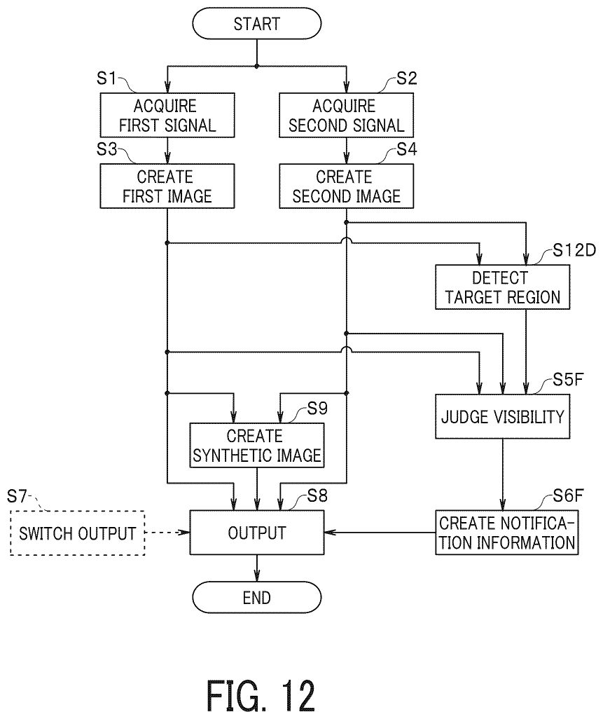

FIG. 12 is a flowchart showing specific processing of an endoscopic image processing device 3 in the third embodiment.

When the processing starts, the processing in the above-described Steps S1 to S4, and S12D is performed.

The visibility judgment section 3d determines whether the first visibility of the target region in the first image is equal to or higher than the first threshold, or lower than the first threshold. Furthermore, the visibility judgment section 3d determines whether the second visibility of the target region in the second image is equal to or higher than the second threshold, or lower than the second threshold (Step S5F). Here, the visibility judgment section 3d calculates the first visibility and the second visibility based on, for example, at least one of the following: the lesion scores (the first score and the second score) calculated by the above-described AI; the matching degree of the positions of the target regions in the first image and the second image; or the visibility score calculated by the above-described second AI.

If the first visibility is lower than the first threshold and the second visibility is equal to or higher than the second threshold, the image synthesizing section 3f synthesizes the first image and the second image to create the synthetic image (Step S9). At this time, the image synthesizing section 3f performs at least one of the following: synthesizing by setting the synthetic ratio; synthesizing with the synthetic ratio of the first image set higher than the synthetic ratio of the second image; or synthesizing only the target region in the second image with the first image.

As a specific example, if the first visibility in the first image is lower than the first threshold and the second visibility in the second image is equal to or higher than the second threshold, the image synthesizing section 3f sets the synthetic ratio of the first image to be lower than that in the case where the first visibility in the first image is equal to or higher than the first threshold (the visibility in the second image is not considered since the visibility in the first image is high). Note that, in general, the synthetic ratios are normalized so that the equation (r1+r2)=1 is satisfied, where the synthetic ratio of the first image is r1 and the synthetic ratio of the second image is r2. Therefore, when the synthetic ratio of the first image is set to be low, the synthetic ratio of the second image is set to be high.

In addition, in general, the first image (observation image) is more suitable for the observation by the user than the second image (recognition image). In view of this, the image synthesizing section 3f may set the synthetic ratios such that the synthetic ratio r1 of the first image is higher than the synthetic ratio r2 of the second image (i.e., r1>r2).

Furthermore, the image synthesizing section 3f may increase the visibility of the target region while maintaining the easiness of the observation of the first image, by synthesizing only the target region of the second image with the first image.

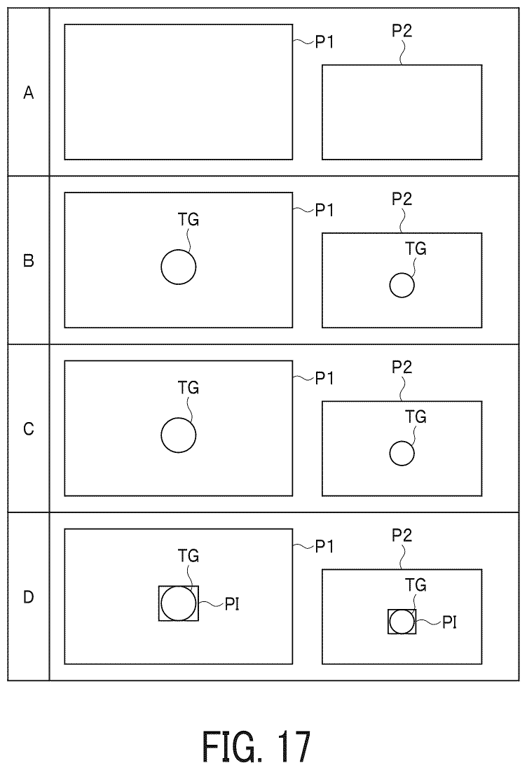

The notification information creation section 3e creates, only when the first visibility is lower than the first threshold and the second visibility is equal to or higher than the second threshold, at least one of: the marker (for example, see the marker PI shown in the section D in FIG. 17) indicating the position of the target region; or the flag (for example, see the flag FG shown in each of FIG. 16, FIG. 18, and FIG. 19) indicating that the target region has been detected. Furthermore, the notification information creation section 3e creates notification information indicating that the image has been switched to the synthetic image with high visibility (see the message MSG2 shown in the section 2B in FIG. 18) (Step S6F).

In the processing in Step S8, only when the first visibility is lower than the first threshold and the second visibility is equal to or higher than the second threshold, the output section 3h outputs to, for example, the one display 4, the synthetic image on which at least one of the marker or the flag is superimposed and the notification information indicating that the image has been switched to the synthetic image.

After Step S8 (further Step S7 as needed) is thus performed, this processing ends.

### Fourth Embodiment

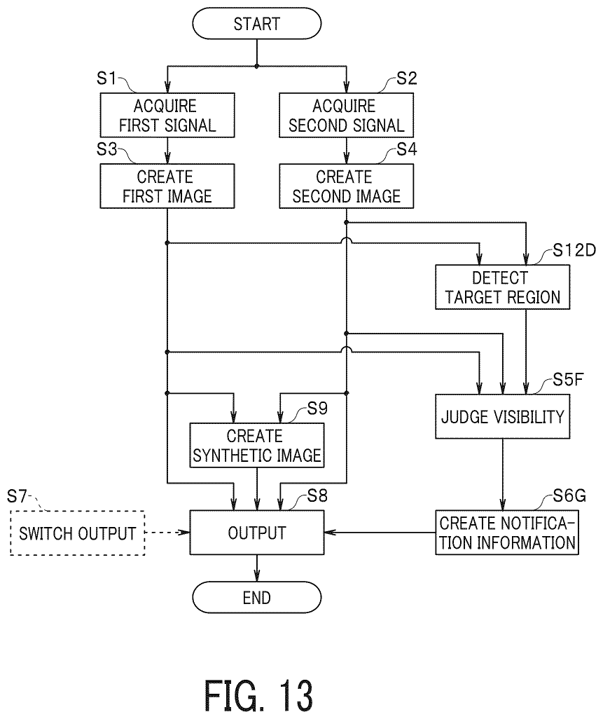

FIG. 13 is a flowchart showing specific processing of an endoscopic image processing device 3 in the fourth embodiment.

When the processing starts, the processing in the above-described Steps S1 to S4, S12D, S5F, and S9 is performed.

The notification information creation section 3e creates, only when the first visibility is lower than the first threshold and the second visibility is equal to or higher than the second threshold, at least one of the marker indicating the position of the target region or the flag indicating that the target region has been detected (Step S6G). At this time, the notification information creation section 3e may set a transparency of the marker to be lower as the first visibility is lower than the second visibility and a difference between the first visibility and the second visibility becomes larger. In addition, the notification information creation section 3e may create a flag for urging confirmation of the image (that is an image other than the first image, for example, the synthetic image) displayed on the sub screen, for the purpose of displaying the flag on the first image.

In the processing in Step S8, if the first visibility is lower than the first threshold and the second visibility is equal to or higher than the second threshold, the output section 3h outputs the first image on which at least one of the marker or the flag is superimposed to the main screen and outputs the synthetic image to the sub screen. Accordingly, by viewing the flag, the user is urged to move his/her line of sight from the main screen to the sub screen.

After Step S8 (further Step S7 as needed) is thus performed, this processing ends.

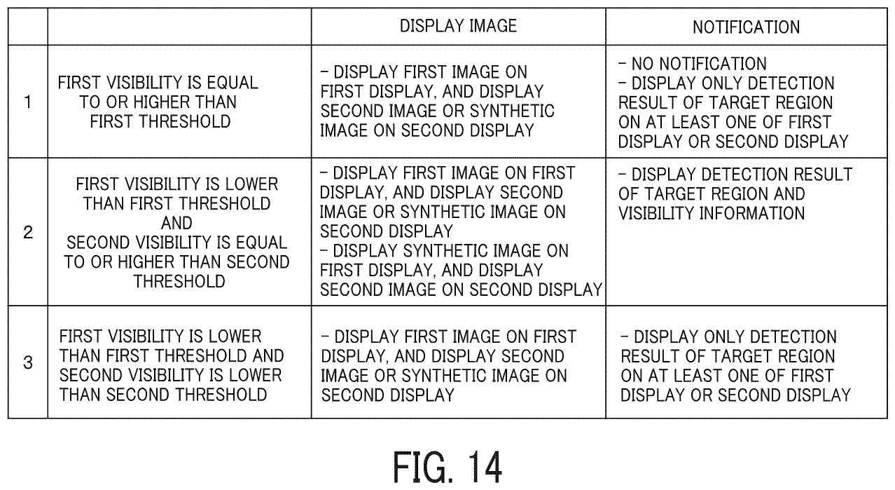

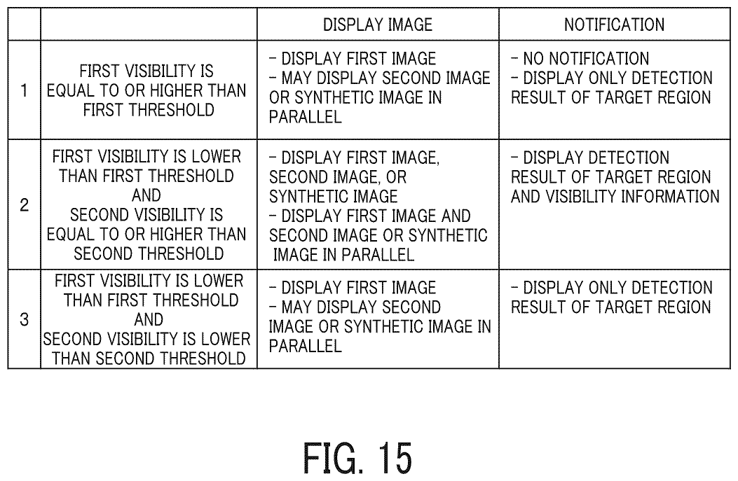

FIG. 14 is a chart showing an example of how the image display and the notification are performed on the first display 4A and the second display 4B depending on the visibility, in each embodiment. Note that, in the description of FIG. 14 and the description of FIG. 15 to be described later, an example is described in which the notification is performed by displaying the visual information. However, the notification may be performed by using the information (the sound information, the voice information, the vibration information, etc.) other than the visual information, as described above.

As shown in the section 1 in FIG. 14, when the first visibility is equal to or higher than the first threshold, it is easy to distinguish the target region such as the lesion candidate region if viewing the first image. In this case, the image display and the notification are performed as described below, for example, regardless of whether the second visibility is equal to or higher than the second threshold or lower than the second threshold.

The first image is displayed on the first display 4A and the second image or the synthetic image is displayed on the second display 4B. In addition, notification does not have to be performed. Alternatively, only the detection result of the target region (markers PI, PI′, etc., to be described later) may be displayed on at least one of the first display 4A or the second display 4B.

As shown in the section 2 in FIG. 14, when the first visibility is lower than the first threshold and the second visibility is equal to or higher than the second threshold, it is difficult to distinguish the target region even if the user views the first image, but it is easy to distinguish the target region if the user views the second image.

In this case, the first image is displayed on the first display 4A and the second image or the synthetic image is displayed on the second display 4B. Alternatively, the synthetic image may be displayed on the first display 4A and the second image may be displayed on the second display 4B. In addition, the detection result of the target region and visibility information are displayed.

As shown in the section 3 in FIG. 14, when the first visibility is lower than the first threshold and the second visibility is lower than the second threshold, it is difficult to distinguish the target region even if the user views either the first image or the second image.

In this case, the first image is displayed on the first display 4A and the second image or the synthetic image is displayed on the second display 4B. In addition, only the detection result of the target region is displayed on at least one of the first display 4A or the second display 4B.

FIG. 15 is a chart showing an example of how the image display and the notification are performed on the one display 4 depending on the visibility, in each embodiment.

As shown in the section 1 in FIG. 15, when the first visibility is equal to or higher than the first threshold, the first image is displayed on the display 4. Furthermore, in addition to the first image, the second image or the synthetic image may be displayed in parallel on the display 4. The notification does not have to be performed. Alternatively, only the detection result of the target region may be displayed.

As shown in the section 2 in FIG. 15, when the first visibility is lower than the first threshold and the second visibility is equal to or higher than the second threshold, the first image, the second image, or the synthetic image is displayed on the display 4. Note that the first image, and the second image or the synthetic image may be displayed in parallel on the display 4. Furthermore, the detection result of the target region and the visibility information are displayed.

As shown in the section 3 in FIG. 15, when the first visibility is lower than the first threshold and the second visibility is lower than the second threshold, the first image is displayed on the display 4. Note that, in addition to the first image, the second image or the synthetic image may be displayed in parallel on the display 4. Furthermore, only the detection result of the target region is displayed.

Note that the image display and the notification are not limited to the examples shown in FIG. 14 and FIG. 15, and other image display and other notification may be performed.

FIG. 16 is a chart showing one example of a display change on the display 4 when an endoscopic examination is being performed, in each embodiment.

When the endoscopic examination is started, the endoscopic image processing device 3 acquires the first signal and the second signal from the endoscope 2, to create a first image P1 and a second image P2. The created first image P1 and second image P2 are displayed side by side on the one display 4 as shown in the section A in FIG. 16, or displayed respectively on the first display 4A and the second display 4B.

The target region detection section 3c detects a target region TG, as shown in the section B in FIG. 16, in each of the first image P1 and the second image P2. The visibility judgment section 3d judges visibility of each of the detected target regions TG.

If the first visibility of the target region TG in the first image P1 is equal to or higher than the first threshold, or the first visibility is lower than the first threshold and the second visibility of the target region TG in the second image P2 is lower than the second threshold, as shown in the section C in FIG. 16, the marker PI (position information) indicating the position of the target region TG is displayed on each of the first image P1 on the main screen and the second image P2 on sub screen. Here, an icon displayed as a quadrangular frame shape surrounding the target region TG is supposed to be the marker PI. However, the marker PI may be displayed in another form.

Note that the shape of the icon indicating the marker PI may be changed depending on the level of the visibility (at least one of the first visibility or the second visibility). In the example shown in the section C in FIG. 16, the marker PI is displayed both on the first image P1 and the second image P2. However, the marker PI may be displayed only on either the first image P1 or the second image P2.

In addition, when the first visibility is lower than the first threshold and the second visibility is equal to or higher than the second threshold, as shown in the section D in FIG. 16, the marker PI indicating the position of the target region TG and the flag FG are displayed on the main screen. Here, the flag FG is used for urging the confirmation of the second image displayed on the sub screen. The marker PI is displayed on the sub screen.

The flag FG is for urging the user to move his/her line of sight from the main screen on which the first image is displayed to the sub screen on which the image other than the first image is displayed. To this end, the flag FG is displayed only on the main screen.

The flag FG is, for example, displayed as triangular figures (icons), by four corners of the first image P1 being painted out with a specific color (yellow, for example). However, the flag FG may be displayed in another form.

FIG. 17 is a chart showing another example of the display change on the display 4 when the endoscopic examination is being performed, in each embodiment.

When the endoscopic examination is started, as shown in the section A in FIG. 17 (similarly as shown in the section A in FIG. 16), the first image P1 and the second image P2 are displayed side by side on the one display 4, or displayed respectively on the first display 4A and the second display 4B.

As shown in the section B in FIG. 17 (similarly as shown in the section B in FIG. 16), the target region detection section 3c detects the target regions TG, and the visibility judgment section 3d judges the visibility of each of the target regions TG.

If the first visibility is equal to or higher than the first threshold, the user can recognize the target region TG by viewing the first image P1. For this reason, regardless of the second visibility, the notification information is displayed neither on the first image P1 nor on the second image P2, as shown in the section C in FIG. 17.

In addition, if the first visibility is lower than the first threshold and the second visibility is equal to or higher than the second threshold, as shown in the section D in FIG. 17, the marker PI indicating the position of the target region TG is displayed on each of the first image P1 on the main screen and the second image P2 on the sub screen.

FIG. 18 is a chart showing a display example of an image depending on the visibility when one image is displayed on the display 4 in each embodiment. In each of the sections 1 and 2 in FIG. 18, the section A shows an example in which the first visibility is equal to or higher than the first threshold, and the section B shows an example in which the first visibility is lower than the first threshold and the second visibility is equal to or higher than the second threshold.

The section 1 in FIG. 18 shows a first display example. If the first visibility is high, the first image P1 is displayed on the display 4 as shown in the section 1A in FIG. 18. The flag FG is superimposed on the first image P1. Since the first visibility is high, in this case, the flag FG is used for notifying that the observation of the first image PI may be continued. Furthermore, the flag FG may be used for notifying that the target is in the image P1.

The flag FG is, for example, displayed as triangular figures, by the four corners of the first image P1 being painted out with a specific color (green, for example). Thus, the flag FG notifying that the observation of the first image P1 may be continued is shown in the color different from that of the flag FG for urging the confirmation of the sub screen on which the image other than the first image is displayed which is shown in the section D in FIG. 16.

As shown in the section 1B in FIG. 18, if the first visibility is low and the second visibility is high, the first image P1 is displayed on the display 4. On the first image P1, the flag FG and the message MSG1 are superimposed. Since the first visibility is low, the flag FG is used here for urging the confirmation of the image other than the first image P1.

The flag FG is, for example, displayed as triangular figures, by the four corners of the first image P1 being painted out with the specific color (green, for example), and by the border lines BL of the flag FG being shown with another specific color (red, for example). Thus, the flag FG used for urging the confirmation of the image other than the first image P1 is made different in the color of the border lines BL from the flag FG notifying that the observation of the first image P1 may be continued shown in the section 1A in FIG. 18.

The message MSG1 is configured as the notification information displayed with characters “Please switch to special light”, for example. Having viewed the message MSG1, the user switches the illumination light from the normal light to the special light, which causes the image displayed on the display 4 to switch from the first image (normal light image) to the second image (special light image). However, the message MSG1 may be displayed with an icon instead of or along with the information with the characters.

The section 2 in FIG. 18 shows a second display example. If the first visibility is high, as shown in the section 2A in FIG. 18, the first image P1 is displayed on the display 4. On the first image P1, the marker PI indicating the position of the target region is superimposed. Here, the marker PI is displayed as a quadrangular frame. If the first visibility is equal to or higher than the first threshold, the marker PI is displayed, for example, as the quadrangular frame surrounded by lines in a specific color (green, for example).

If the first visibility is low and the second visibility is high, as shown in the section 2B in FIG. 18, a synthetic image PS is displayed on the display 4. On the synthetic image PS, a marker PI′ indicating the position of the target region and the message MSG2 are superimposed. If the first visibility is lower than the first threshold, the marker PI′ is displayed as a quadrangular frame surrounded by lines in another specific color (red, for example). Thus, the marker PI′ indicating that the first visibility is low is made different in the color of the lines from the marker PI indicating that the first visibility is high shown in the section 2A in FIG. 18.

The message MSG2 is configured as the notification information with characters “Switched to synthetic image”, for example. Note that the message MSG2 may be displayed with an icon instead of or along with the information with the characters. Having viewed the message MSG2, the user can recognize that the image displayed on the display 4 has automatically switched from the first image (normal light image) to the synthetic image with high visibility.

FIG. 19 is a chart showing a display example of images depending on the visibility when two images are displayed respectively on the main screen and the sub screen, in each embodiment. Note that, in FIG. 19, the screen displayed on the left side and displaying the image in a large size is the main screen, and the screen displayed on the right side and displaying the image in a size smaller than the size of the image on the main screen is the sub screen.

In each of the sections 1 to 3 in FIG. 19, the section A shows an example in which the first visibility is equal to or higher than the first threshold, and the section B shows an example in which the first visibility is lower than the first threshold and the second visibility is equal to or higher than the second threshold.

The section 1 in FIG. 19 shows a first display example. If the first visibility is high, the first image P1 is displayed on the main screen and the second image P2 is displayed on the sub screen, as shown in the section 1A in FIG. 19. The marker PI indicating the position of the target region is superimposed on each of the first image P1 and the second image P2. Similarly as shown in the section 2A in FIG. 18, if the first visibility is equal to or higher than the first threshold, the marker PI is displayed, for example, as the quadrangular frame surrounded by the lines in the specific color (green, for example).

If the first visibility is low and the second visibility is high, the first image P1 is displayed on the main screen and the second image P2 is displayed on the sub screen, as shown in the section 1B in FIG. 19. On the first image P1, the marker PI′ indicating the position of the target region and the message MSG3 are superimposed. On the second image P2, the marker PI′ indicating the position of the target region is superimposed.

Similarly as shown in the section 2B in FIG. 18, if the first visibility is lower than the first threshold, the marker PI′ is displayed as the quadrangular frame surrounded by the lines in the other specific color (red, for example). Thus, the marker PI′ indicating that the first visibility is low is made different in the color of the lines from the marker PI indicating that the first visibility is high shown in the section 1A in FIG. 19.

The message MSG3 is configured as the notification information with characters “Please confirm sub screen”, for example. Note that the message MSG3 may be displayed with an icon instead of or along with the information with the characters. Having viewed the message MSG3, the user can recognize that he or she is urged to move his/her line of sight from the main screen to the sub screen.

The section 2 in FIG. 19 shows a second display example. If the first visibility is high, the first image P1 is displayed on the main screen and the second image P2 is displayed on the sub screen, as shown in the section 2A in FIG. 19. Since the first visibility is high, the flag FG indicating that the observation of the first image P1 may be continued is superimposed on the first image P1. Furthermore, the flag FG may be used for notifying that the target is in the image P1. The flag FG is, for example, displayed as triangular figures by the four corners of the first image P1 being painted out with the specific color (green, for example), similar to the flag FG shown in the section 1A in FIG. 18.

The marker PI indicating the position of the target region is superimposed on the second image P2. Similarly as shown in the section 1A in FIG. 19, if the first visibility is equal to or higher than the first threshold, the marker PI is displayed, for example, as the quadrangular frame surrounded by the lines in the specific color (green, for example).

If the first visibility is low and the second visibility is high, the first image P1 is displayed on the main screen and the second image P2 is displayed on the sub screen, as shown in the section 2B in FIG. 19. On the first image P1, the flag FG for urging the confirmation of the sub screen and the message MSG3 are superimposed.

The flag FG is, for example, displayed as triangular figures by the four corners of the first image P1 being painted out with another specific color (yellow, for example). Thus, the flag FG for urging the confirmation of the sub screen is made different in the color from the flag FG shown in the section 2A in FIG. 19. The message MSG3 is the same as the message MSG3 shown in the section 1B in FIG. 19.

On the second image P2, the marker PI′ indicating the position of the target region is superimposed. Similarly as shown in the section 1B in FIG. 19, if the first visibility is lower than the first threshold, the marker PI′ is displayed, for example, as the quadrangular frame surrounded by the lines in the other specific color (red, for example).

The section 3 in FIG. 19 shows a third display example. If the first visibility is high, the first image P1 is displayed on the main screen and the synthetic image PS is displayed on the sub screen, as shown in the section 3A in FIG. 19. Since the first visibility is high, the flag FG notifying that the observation of the first image P1 may be continued is superimposed on the first image P1. Furthermore, the flag FG may be used for notifying that the target is in the image P1. The flag FG is the same as the one shown on the main screen in the section 2A in FIG. 19.

On the synthetic image PS, the marker PI indicating the position of the target region is superimposed. The marker PI is the green quadrangular frame which is the same as the marker PI shown on the sub screen in the section 2A in FIG. 19.

If the first visibility is low and the second visibility is high, the first image P1 is displayed on the main screen and the synthetic image PS is displayed on the sub screen, as shown in the section 3B in FIG. 19. On the first image P1, the flag FG for urging the confirmation of the sub screen and the message MSG3 are superimposed. The flag FG and the message MSG3 are the same as those shown on the main screen in the section 2B in FIG. 19.

On the synthetic image PS, the marker PI′ indicating the position of the target region is superimposed. The marker PI′ is the red quadrangular frame which is the same as the marker PI′ shown on the sub screen in the section 2B in FIG. 19.

Note that, among the various kinds of notification information, the visual information to be displayed on the display 4 includes the markers PI and PI′, the flags FG, the messages MSG1 to MSG3, and the like. Among these, the markers PI and PI′ each indicating the position of the target region may be displayed in an endoscopic image. On the other hand, the flags FG and the messages MSG1 to MSG3 may be displayed outside the endoscopic image, as long as they are displayed within the display screen of the display 4.

According to the respective embodiments as described above, the line of sight of the user is guided to the second image or the image is switched to the second image, only when the second image in which the target region is easily distinguishable with the human eyes is created. Such a configuration prevents the user from meaninglessly moving his/her line of sight to the second image in which the target region is hard to distinguish (or prevents the image from being meaninglessly switched to the second image). This can shorten the time for the endoscopic examination as much as possible, reduce a burden on a patient, and improve the examination efficiency.

In addition, the notification information urging caution is created to perform notification, only when the visibility in the first image is low and the visibility in the second image is high. Such a configuration enables the target region, the visual recognition of which is difficult in the first image, to be found by observing the second image with high visibility, which reduces overlooking of the target region.

Thus, the notification information for enabling the user to easily visually recognize at least a part of the image can be created.

Note that the present disclosure is described mainly by taking the case where the present disclosure is the endoscopic image processing device as an example. However, the present disclosure is not limited to the case. For example, the present disclosure may be an endoscope system including the endoscopic image processing device. The present disclosure may be the method for operating the endoscopic image processing device, as described above. The present disclosure may be a computer program for causing a computer to perform the same processing as that performed by the endoscopic image processing device. The present disclosure may be a non-transitory computer-readable recording medium that records the computer program, or other media.

Some examples of the recording medium that stores a computer program product include a portable recording medium such as a flexible disk, a compact disc read only memory (CD-ROM), a digital versatile disc (DVD) or the like, or a recording medium such as a hard disk. The flexible disk, the CD-ROM, the DVD, and the hard disk are examples of the non-volatile storage medium. It is not limited to the entirety of the computer program to be stored in the recording medium, but a part of the computer program may be stored in the recording medium. In addition, the entirety or a part of the computer program may also be distributed or provided via a communication network. When a user installs the computer program on the computer from the recording medium or downloads the computer program via the communication network and installs it on the computer, the computer program is read by the computer and all or part of the operations are performed, to thereby enable the operations of the endoscopic image processing device as described above to be performed.

Furthermore, the present invention is not limited as-is to the above described embodiments. It is possible to embody the present disclosure by modifying the constituent elements in a range without departing from the gist of the invention at the practical stage. In addition, various aspects of the invention can be achieved by appropriately combining the plurality of constituent elements disclosed in the above-described embodiment. Some of the constituent elements may be deleted from all the constituent elements disclosed in the embodiment, for example. Furthermore, constituent elements over different embodiments may be combined as appropriate. It goes without saying that various modifications and applications can be implemented within a range without departing from the gist of the invention.

What is claimed is:

1. An endoscopic image processing device comprising one or more processors,
the one or more processors being configured to:
acquire a first signal related to an image with a first condition;
acquire a second signal related to an image with a second condition different from the first condition;
create a first image from the first signal;
create a second image from the second signal;
determine whether visibility of at least a part of the second image is equal to or higher than a second threshold, or lower than the second threshold; and
create different notification information depending on whether the visibility is equal to or higher than the second threshold, or lower than the second threshold,
wherein the one or more processors create at least one kind of:
the notification information for guiding to the second image when the visibility is equal to or higher than the second threshold; or
the notification information for notifying that the second image is not suitable for viewing when the visibility is lower than the second threshold.

2. The endoscopic image processing device according to claim 1, wherein
the one or more processors are configured to:
acquire a signal for detection;
detect a target region based on the signal for detection; and
determine whether visibility of the target region in the second image is equal to or higher than the second threshold, or lower than the second threshold.

3. The endoscopic image processing device according to claim 2, wherein
the second signal serves also as the signal for detection, and
the one or more processors are configured to detect the target region from the second image.

4. The endoscopic image processing device according to claim 3, wherein
when a threshold for the target region in the first image is defined as a first threshold,
the one or more processors are further configured to:
determine whether first visibility of the target region in the first image is equal to or higher than the first threshold, or lower than the first threshold; and
create, when the first visibility is lower than the first threshold, the notification information that is different depending on whether second visibility of the target region in the second image is equal to or higher than the second threshold, or lower than the second threshold.

5. The endoscopic image processing device according to claim 4, wherein
the one or more processors are configured to determine whether the second visibility is equal to or higher than the second threshold, or lower than the second threshold, based on information on the target region in the second image and information on a region outside the target region in the second image.

6. The endoscopic image processing device according to claim 5, wherein
the one or more processors are configured to determine whether the first visibility is equal to or higher than the first threshold, or lower than the first threshold, based on information on the target region in the first image and information on a region outside the target region in the first image.

7. The endoscopic image processing device according to claim 6, wherein
the one or more processors are configured to:
calculate a first color difference between the target region and the region outside the target region in the first image;
compare the first color difference with the first threshold by using the first color difference as the first visibility;
calculate a second color difference between the target region and the region outside the target region in the second image; and
compare the second color difference with the second threshold by using the second color difference as the second visibility.

8. The endoscopic image processing device according to claim 6, wherein
the one or more processors are configured to:
calculate a first blood vessel amount difference between the target region and the region outside the target region in the first image;
compare the first blood vessel amount difference with the first threshold by using the first blood vessel amount difference as the first visibility;
calculate a second blood vessel amount difference between the target region and the region outside the target region in the second image; and
compare the second blood vessel amount difference with the second threshold by using the second blood vessel amount difference as the second visibility.

9. The endoscopic image processing device according to claim 4, wherein
the one or more processors are configured to:
further detect the target region from the first image;
further detect a first score indicating accuracy of the target region in the first image, and a second score indicating accuracy of the target region in the second image;
compare the first score with the first threshold by using the first score as the first visibility; and
compare the second score with the second threshold by using the second score as the second visibility.

10. The endoscopic image processing device according to claim 4, wherein
the one or more processors are configured to:
further detect the target region from the first image;
determine that the first visibility is equal to or higher than the first threshold when the target region is detected from the first image, and determine that the first visibility is lower than the first threshold when the target region is not detected; and
determine that the second visibility is equal to or higher than the second threshold when the target region is detected from the second image, and determine that the second visibility is lower than the second threshold when the target region is not detected.

11. The endoscopic image processing device according to claim 2, wherein
the one or more processors are configured to create an icon indicating a position of the target region, and
output the icon along with the first image to a display.

12. The endoscopic image processing device according to claim 2, wherein
the target region is a lesion candidate region.

13. The endoscopic image processing device according to claim 2, wherein
the one or more processors are configured to synthesize the first image and the second image to create a synthetic image, and
output the synthetic image to a display.

14. The endoscopic image processing device according to claim 13, wherein
the one or more processors are configured to set a synthetic ratio of the first image and the second image, to create the synthetic image.

15. The endoscopic image processing device according to claim 13, wherein
the one or more processors are configured to synthesize the target region in the second image with first image, to create the synthetic image.

16. A method for operating an endoscopic image processing device comprising one or more processors,
the one or more processors being configured to:
acquire a first signal related to an image with a first condition;
acquire a second signal related to an image with a second condition different from the first condition;
output, to one or more displays, a first image created from the first signal, and a second image created from the second signal;
determine whether visibility of at least a part of the second image is equal to or higher than a second threshold, or lower than the second threshold; and
create notification information for guiding to the second image when the visibility is equal to or higher than the second threshold, or
create notification information for notifying that the second image is not suitable for viewing when the visibility is lower than the second threshold.

17. A non—volatile storage medium that stores a computer program for operating an endoscopic image processing device,
the program causing one or more processors to perform processing of:
acquiring a first signal related to an image with a first condition;
acquiring a second signal related to an image with a second condition different from the first condition;
outputting, to one or more displays, a first image created from the first signal, and a second image created from the second signal;
determining whether visibility of at least a part of the second image is equal to or higher than a second threshold, or lower than the second threshold; and
creating notification information for guiding to the second image when the visibility is equal to or higher than the second threshold, or
creating notification information for notifying that the second image is not suitable for viewing when the visibility is lower than the second threshold.
